(19) United States
(12) Patent Application Publication
(10) Pub. No.: US 2007/0042015 A1
(43) Pub. Date: Feb. 22, 2007

(54) Coating composition for polymeric surfaces comprising serpin or serpin derivatives

(76) Inventors: Leslie Roy Berry, Ontario (CA); Anthony Kam Chuen Chan, Ontario (CA); Ying Jun Du, Acton, MA (US); Paul Tressel, Ontario (CA)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/546,242

(22) PCT Filed: Feb. 20, 2004
(86) PCT No.: PCT/CA04/00247

§ 371 Date: Aug. 7, 2006

Related U.S. Application Data
(60) Provisional application No. 60/448,116, filed on Feb. 20, 2003.

(51) Int. Cl. A61K 6/083; A61K 31/727

(52) U.S. Cl. 424/423; 514/56; 427/2.26

(57) ABSTRACT

The invention relates generally to a coating composition for a polymeric surface, methods for coating a polymeric surface, methods for preparing coated medical devices, polymeric surfaces coated with the coating composition, and medical devices comprising the coating composition. In particular, a coating composition for association with a polymeric surface, preferably a polymeric surface of a medical device, is described comprising a cross-linked basecoat displaying a plurality of active groups in association with a serpin or serpin derivatives, wherein the serpin or serpin derivatives are not substantially cross-linked with other serpin or serpin derivatives.

### FIELD OF THE INVENTION

The invention relates generally to a coating composition for a polymeric surface, methods for coating a polymeric surface, methods for preparing coated medical devices, polymeric surfaces coated with the coating composition, and medical devices comprising the coating.

### BACKGROUND OF THE INVENTION

Clotting is a significant clinical issue for many devices including hemodialysis catheters, central venous access catheters, endoluminal grafts, coronary and peripheral stents, extracorporeal devices, etc. The physical consequences of clotting in a device can be very serious and can ultimately lead to pulmonary embolism if the clot dislodges and travels to the lung. In addition to the physical damage caused by the clot and the distress experienced by the patient, there are major costs associated with removing clots with thrombolytic drugs or through surgical revisions. Hence various types of surface coatings have been developed for medical devices that are exposed to blood in order to prevent clotting. The most common anticoagulant used for this purpose is heparin.

A covalent complex of antithrombin (AT) and heparin (ATH) has been developed that has significant anticoagulant activities (Chan et al. Journal of Biological Chemistry 272:22111-22117, 1997; Chan et al. Blood Coagulation and Fibrinolysis 9:587-595, 1998; Berry et al. Journal of Biological Chemistry 273:34730-34736, 1998; Berry et al. Journal of Biochemistry, 132:167-176, 2002; Klement et al. Biomaterials 23:527-535, 2002; Chan et al. Thrombosis and Haemostasis 87:606-613, 2002; Chan et al. Circulation 106:261-265, 2002). ATH was shown to react rapidly with IIa (Chan et al. Journal of Biological Chemistry 272:22111-22117, 1997; Berry et al. Journal of Biological Chemistry 273:34730-34736, 1998; Chan et al. Circulation 106:261-265, 2002) forming a stable covalent ATH-IIa complex (Chan et al. Journal of Biological Chemistry 272:22111-22117, 1997). Furthermore, ATH also possesses a potent ability to catalyze inhibition of factor Xa or IIa by added AT (Chan et al. Journal of Biological Chemistry 272:22111-22117, 1997; Chan et al. Thrombosis and Haemostasis 87:606-613, 2002). ATH has a more rapid onset of action than heparin or antithrombin alone. For antithrombin to bind to, and inactivate thrombin, it must first be rendered active through the binding of heparin through a specific pentasaccharide sequence. In the ATH molecule, antithrombin is already in the active conformation, ready to bind to and inactivate thrombin, thereby inhibiting clot formation. In addition, ATH has improved potency over heparin because all of the heparin chains in ATH are active (Berry et al. Journal of Biological Chemistry 273:34730-34736, 1998).

ATH coated devices and processes for coating medical devices with ATH are described in U.S. Pat. No. 6,491,965, in Klement et al. Biomaterials 23:527-535, 2002 and in Berry L., Andrew M. and Chan A. K. C. Antithrombin-Heparin Complexes (Chapter 25). In: Polymeric Biomaterials. Part II: Medical and Pharmaceutical Applications of Polymers. (Second Edition) Ed. S. Dumitriu. Marcel Dekker Inc., New York, pp. 669-702, 2001.

The citation of any reference herein is not and admission that such reference is available as prior art to the instant invention.

### SUMMARY OF THE INVENTION

The present invention provides improved coating compositions and methods for coating polymeric surfaces. In an aspect, the invention provides improved coated polymeric surfaces, in particular, medical devices. More specifically, the present invention provides improved medical devices, and methods of manufacturing same.

The advantages achieved by the present invention include a simple and readily controlled process that can provide improved coating compositions that can be used with suitable substrates, in particular medical devices. Moreover, a method of the invention provides a stable attachment of a coating to a polymeric surface, in particular, a polymeric surface of a medical device. The invention can provide a permanent coating technique that assures more uniform coverage of a polymeric surface. It also allows surface modification of devices to provide advantageous properties such as anti-thrombogenic properties. The invention can provide greater exposure of active or therapeutic compounds in the coating to biological fluids or surfaces that are in contact with the coating. For example, it can provide greater exposure of anticoagulants in the coating to blood.

Therefore, in an aspect, the invention relates to a coating composition for association with a polymeric surface, preferably a polymeric surface of a suitable substrate, in particular a medical device, comprising a cross-linked basecoat displaying a plurality of active groups in association with serpins or serpin derivatives, wherein the serpins or serpin derivatives are not substantially cross-linked with other serpins or serpin derivatives. The coating composition may be in association or combination with a polymeric surface.

In another aspect, the invention provides a method for coating a polymeric surface with a serpin or serpin derivative which comprises the following steps:
(i) introducing monomers, preferably heterofunctional monomers, with active groups on the polymeric surface; and
(ii) reacting with a preparation comprising the serpin or serpin derivative so that the serpin or serpin derivative associates with the active groups.

The invention also provides a polymeric surface that is coated with a basecoat displaying a plurality of active groups associated with serpins or serpin derivatives.

The invention also contemplates a coated polymeric surface prepared by a method of the invention.

The invention also relates to a suitable substrate for incorporating a coating composition of the invention, in particular a medical device.

The invention contemplates a medical device comprising a polymeric surface that is coated with a coating composition comprising a cross-linked basecoat displaying a plurality of active groups in association with serpins or serpin derivatives, wherein serpins or serpin derivatives are not substantially cross-linked with serpins or serpin derivatives.

The invention further contemplates a method for preparing a coated medical device comprising coating a polymeric surface of the medical device with a composition of the invention.

The invention also relates to a kit for preparing a coating composition, a coated polymeric surface, or a coated medical device according to the invention.

The present invention additionally provides methods of rendering a blood- or tissue-contacting surface of a medical device resistant to fibrin accumulation and/or clot formation which method comprises coating at least a portion of a polymeric surface of the medical device with a coating composition of the invention.

The invention further contemplates a method of rendering a polymeric surface of a preformed medical material or device anti-thrombogenic comprising coating the polymeric surface with a coating composition of the invention.

In another aspect the invention contemplates a method of rendering a polymeric surface of a preformed medical material or device anti-thrombogenic comprising coating the polymeric surface with a coating composition of the invention.

A coating composition of the invention may be used to reduce clotting in a medical device used in a patient. Therefore, the invention provides a method of treating a patient comprising introducing into the patient a medical device comprising a polymeric surface coated with a coating composition of the invention in an amount sufficient to prevent or inhibit thrombosis.

The present invention additionally provides methods of using or uses of a medical device coated with a coating composition of the invention. In an embodiment, the use or method comprises providing to a patient in need thereof a medical device comprising a body and at least a portion of the body coated with a coating composition comprising a cross-linked basecoat displaying a plurality of active groups capable of associating with serpins or serpin derivatives, wherein the serpins or serpin derivatives are not substantially cross-linked with other serpins or serpin derivatives.

These and other aspects, features, and advantages of the present invention should be apparent to those skilled in the art from the following drawings and detailed description.

### DESCRIPTION OF THE DRAWINGS

The invention will be better understood with reference to the drawing in which:

FIG. 1 shows a schematic diagram of a method for covalent linkage to a polymeric surface of a basecoat displaying a plurality of active groups in association with an antithrombin-heparin complex.

FIG. 2 shows a schematic diagram of a method for non-covalent linkage to a polymeric surface of a basecoat displaying a plurality of active groups in association with an antithrombin-heparin complex

FIG. 3 shows immunoblots of proteins eluted from the inner (I) and outer (O) surfaces of PU-ATH catheters following in vivo experiments.

FIG. 4 shows the effects of monomer composition and total monomer concentration on ATH graft density of coated catheters during washing with saline (0.8 g NaCl/100 ml H₂O). Saline washing solution was replaced every 24 hours with fresh saline washing solution and the catheters analyzed for ¹²⁵I-ATH. Codes are for monomer composition and total concentration. For example, 112-20 is an experiment in which the ratio of volume of poly(ethyleneglycol)diacrylate monomer to volume of isocyanato-ethylmethacrylate monomer to volume of glycidyl methacrylate monomer in the basecoat is 1 to 1 to 2 and the percent of total volume of all monomers in the total volume of monomers+solvent is 20. 103-20 is an experiment in which the ratio of volume of glycidyl methacrylate to polyethyleneglycol methacrylate in the basecoat is 3:1.

FIG. 5 shows the effects of monomer composition and total monomer concentration on ATH graft density of coated catheters during washing with sodium dodecyl sulfate (2 g SDS/100 ml H₂O). SDS washing solution was replaced every 24 hours with fresh SDS washing solution and the catheters analyzed for ¹²⁵I-ATH. Codes are for monomer composition and total concentration. For example, 112-20 is an experiment in which the ratio of volume of poly(ethyleneglycol)diacrylate monomer to volume of isocyanato-ethylmethacrylate monomer to volume of glycidyl methacrylate monomer in the basecoat is 1 to 1 to 2 and the percent of total volume of all monomers in the total volume of monomers+solvent is 20. The experiment designated as contl was a control experiment in which a catheter that was not coated with a basecoat was incubated with ¹²⁵I-ATH, followed by the same washing procedure.

FIG. 6 shows the effects of monomer composition and total monomer concentration on ATH graft density of coated catheters 0.1 mg of protease/ml. Protease solution was replaced with fresh protease solution every 24 hours and the catheters analyzed for ¹²⁵I-ATH. Codes are for monomer composition and total concentration. For example, 112-20 is an experiment in which the ratio of volume of poly(ethyleneglycol)diacrylate monomer to volume of isocyanato-ethylmethacrylate monomer to volume of glycidyl methacrylate monomer in the basecoat is 1 to 1 to 2 and the percent of total volume of all monomers in the total volume of monomers+solvent is 20.

### DETAILED DESCRIPTION OF THE INVENTION

Glossary

“Coating” or “coated” in the context of a method, of the invention refers to complete, substantially complete; or partial coverage of a polymeric surface with a cross-linked basecoat displaying a plurality of active groups associated with a serpin or serpin derivative. In embodiments of the invention at least a portion of the polymeric surface is covered with a basecoat.

The term “associate”, “association” or “associating” refers to a condition of proximity between an active group and a serpin or serpin derivative, or parts or fragments thereof, or between a polymeric surface and a cross-linked basecoat displaying a plurality of active groups associated with a serpin or serpin derivative or a coating composition of the invention. The association may be non-covalent i.e. where the juxtaposition is energetically favored by for example, hydrogen-bonding, van der Waals, or electrostatic or hydrophobic interactions, or it may be covalent.

“Polymeric surface” refers to a surface that is capable of being coated with a serpin or serpin derivative or coating composition in accordance with a method of the invention. A polymeric surface may be natural or synthetic.

A polymeric surface may be composed of a synthetic polymer. A synthetic polymer may be composed of urethanes, acrylates, acrylamides, (for example, polyurethanes, polyacrylates, and polymethacrylates), and combinations thereof. Examples of particular polymers include but are not limited to poly 2-hydroxyethyl methacrylate, polyacrylamide, polyether polyurethane urea (PEUU), polyurethane, silicone, polyethylene, polypropylene, polytetrafluoroethylene, poly(vinylchloride), polydimethylsiloxane, an ethylene-acrylic acid copolymer, Dacron, polyester-polyurethane, polycarbonate-polyurethane, urethane acrylate, epoxy acrylate, polyamide (Nylon) and polystyrene.

A polymeric surface may be a surface of a suitable substrate, in particular a medical device. As used herein, “medical device” refers to any material comprising a polymeric surface that is used in the treatment, monitoring, or prophylaxis of a condition in a patient. The device is preferably one that is implanted into a patient or otherwise comes into contact with blood and for which it would be desirable to reduce blood coagulation. In an embodiment, the device is suited for introduction into the coronary and peripheral vascular systems.

Examples of medical devices include catheters, multilumen catheters, drip chamber filter meshes for blood circuits employed for extracorporeal circulation, film or hollow fibre oxygen-exchanging membranes for artificial lungs and connectors for tube connections, film or hollow fibre dialysis membranes for artificial kidneys, endovascular tubing, arterial and central venous lines, cardiac catheters, cardiopulmonary bypass circuits, dialysis circuits, wound drains, guide wires, nerve-growth guides, chest tubes, septums, hemodialysis catheters, central venous access catheters, endoluminal grafts, stents including coronary and peripheral stents, AV shunts for artificial kidneys and artificial blood vessels, sheath introducers, canulas, by-pass tubes, extracorporeal devices or other external blood contacting instruments, as well as pacemaker leads, arterial and venous catheters for cannulation of large vessels thrombectomy catheters, sutures, blood filters, intravenous lines, mechanical valves, stents, prosthetics, cardiovascular grafts, bone replacements, wound healing devices, cartilage replacement devices, urinary tract replacements, artificial kidneys, lungs, hearts, heart valves, and livers or any in vivo prosthesis, especially those made from a natural or synthetic polymer or polymers. Other examples of medical devices that would benefit from the application of a coating composition of the invention will be readily apparent to those skilled in the art of surgical and medical procedures and are therefore contemplated by the instant invention.

Polymeric surfaces of medical devices may comprise Ioplex materials and other hydrogels such as those based on 2-hydroxyethyl methacrylate or acrylamide, and polyether polyurethane ureas (PEUU) including Biomer (Ethicon Corp.) and Avcothane (Avco-Everrett Laboratories). Materials used most frequently for tubular applications are polyethylene, poly 2-hydroxyethyl methacrylate, polypropylene, silicone, polytetrafluoroethylene (Gore-Tex), poly(vinylchloride), polydimethylsiloxane, an ethylene-acrylic acid copolymer, polycarbonate, polyester, polyamide, polyacrylate, polyvinyl alcohol, polycaprolactone, polylactide, polyglycolide, knitted or woven Dacron, polyester-polyurethane, polyurethane, polycarbonate-polyurethane (Corethane™), vinyl acrylate, allyl compounds, polyamide (Nylon) and polystyrene, and co-polymers of any two or more of the foregoing, siloxanes, natural and artificial rubbers, glass, and metals, including steel and graphite. Additional compounds used in prosthetics and medical devices which come into blood contact are described in Kirk-Othmer Encyclopedia of Chemical Technology, 3rd Edition 1982 (Vol. 19, pp. 275-313, and Vol. 18, pp. 219-2220) and van der Giessen et al., Circulation 94:1690-1997 (1996) both of which are incorporated herein by reference.

A polymeric surface may be associated with a biological tissue such as vascular grafts, heart valve tissues, or synthetic membranes made from various hydrophobic or hydrophilic polymers.

A polymeric surface may be associated with a matrix employed in the fractionation of cells, in particular blood cells. A matrix may be a packing material contained within a column or fibrous material compressed into a filter and held in a housing of conventional design and construction.

“Serpin(s)” refers to a serine protease inhibitor and is exemplified by species comprising antithrombin III and heparin cofactor II. The term includes a serpin derivative. “Serpin derivative” refers to a serpin that possesses a biological activity (either functional or structural or both) that is substantially similar to the biological activity of a serpin. The term “derivative” is intended to include “variants” “analogs” or “chemical derivatives” of a serpin. The term “variant” is meant to refer to a molecule substantially similar in structure and/or function to a serpin or a part thereof. A molecule is “substantially similar” to a serpin if both molecules have substantially similar structures or if both molecules possess similar biological activity. The term “analog” refers to a molecule substantially similar in function to a serpin. The term “chemical derivative” describes a molecule that contains additional chemical moieties that are not normally a part of the base molecule. A serpin may be obtained from natural or non-natural sources (e.g. recombinant or transgenic) and it may be obtained from commercial sources.

“Serpin(s)” also refers to conjugates or complexes comprising a serpin, in particular a conjugate or complex comprising a serpin associated with a glycosaminoglycan.

The term “glycosaminoglycan” refers to linear chains of largely repeating disaccharide units containing a hexosamine and an uronic acid. The precise identity of the hexosamine and uronic acid may vary widely. The disaccharide may be optionally modified by alkylation, acylation, sulfonation (O— or N-sulfated), sulfonylation, phosphorylation, phosphonylation and the like. The degree of such modification can vary and may be on a hydroxyl group or an amino group. Most usually the C6 hydroxyl and the C2 amino are sulfated. The length of the chain may vary and the glycosaminoglycan may have a molecular weight of greater than 200,000 daltons, typically up to 100,000 daltons, and more typically less than 50,000 daltons. Glycosaminoglycans are typically found as mucopolysaccharides. Representative examples of glycosaminoglycans include, heparin, dermatan sulfate, heparan sulfate, chondroitin-6-sulfate, chondroitin-4-sulfate, keratan sulfate, chondroitin, hyaluronic acid, polymers containing N-acetyl monosaccharides (such as N-acetyl neuraminic acid, N-acetyl glucosamine, N-acetyl galactosamine, and N-acetyl muramic acid) and the like and gums such as gum arabic, gum Tragacanth and the like. See Heinegard, D. and Sommarin Y. (1987) Methods in Enzymology 144:319-373. In an embodiment, the glycosaminoglycan is heparin.

In a particular embodiment of the invention, the serpin is antithrombin associated with heparin.

The methods, coating compositions, devices and kits of the present invention preferably use an antithrombin and heparin covalent conjugate (i.e. ATH) as described in U.S. Pat. No. 6,491,965, Klement et al. Biomaterials 23:527-535, 2002 and in Berry L., Andrew M. and Chan A. K. C. Antithrombin-Heparin Complexes (Chapter 25). In: Polymeric Biomaterials. Part II: Medical and Pharmaceutical Applications of Polymers. (Second Edition) Ed. S. Dumitriu. Marcel Dekker Inc., New York, pp. 669-702, 2001. The antithrombin in ATH may be derived from plasma (see for example, U.S. Pat. No. 4,087,415), it may be transgenic (see for example, U.S. Pat. No. 6,441,145), or recombinant (see for example, U.S. Pat. No. 4,632,981). Heparin may be obtained from pig intestine or bovine lung or it may be obtained from commercial sources. Preferably, the heparin is a “high affinity” heparin enriched for species containing more than one copy of the pentasaccharide.

“Not substantially cross-linked” in the context of serpin and serpin derivatives in a coating composition, device, kit, or method of the invention means that the degree of cross-linking of the serpin or serpin derivatives with other serpins or serpin derivatives is less than 1-5%, 1%, 5%, 10%, 15%, 20%, 25%, 30%, and 40%.

A “basecoat” refers to polymers comprising monomers after the monomers have been polymerized. The degree of polymerization of the monomers is typically 50% or more, 60% or more, and particularly 80% and more, and further 90% or more. The degree of polymerization can be substantially 100%.

“Monomers” refers to any compounds with active groups that are capable of polymerizing and associating with a serpin or serpin derivative, in particular compounds with unsaturated double bonds. The monomers may have active groups such as epoxide or epoxy groups. A preparation of the same or different monomers can be used to prepare a coating composition or coat a polymeric surface in accordance with the invention.

The monomers may be heterofunctional monomers of the Formula I:
Aₙ-(R)—Bₙ (Formula I)
Thus, a basecoat may comprise a polymer containing heterofunctional monomers of the Formula I. The “A” group used in the context of a monomer of the Formula I is a group capable of polymerizing. The “B” group used in the context of the Formula I is an active group capable of associating with a serpin or serpin derivative. Suitable “A” and “B” groups include but are not limited to acryloyl, methacryloyl, N-succinimidyl, sulfonylsuccinimidyl, glycidyl ether, 1,2-epoxy, chlorocarbonyl and anhydride. In an embodiment, the B group is glycidyl ether. In the context of the Formula I, “n” is an integer, preferably 1-40, more preferably 1-20, still more preferably 1-10, most preferably 1-5, 1-3, or 1.

The “R” group used in the context of the Formula I is an optional linker. In an aspect R is a hydrocarbyl group, preferably a C₁-C₅₀ divalent hydrocarbyl group.

A “hydrocarbyl group” as used herein in connection with the optional linker R of the Formula I, refers to organic compounds or radicals consisting of the elements carbon and hydrogen. These moieties include alkyl, alkenyl, alkynyl, and aryl moieties. These moieties also include alkyl, alkenyl, alkynyl, and aryl moieties substituted with other aliphatic or cyclic hydrocarbon groups, such as alkaryl, alkenaryl and alkylaryl. Unless otherwise indicated, these moieties preferably comprise 1 to 50 carbon atoms, more preferably 1 to 30 carbon atoms. A hydrocarbyl moiety may be substituted with at least one atom other than carbon, including moieties in which a carbon chain atom is substituted with a hetero atom such as nitrogen, oxygen, silicon, phosphorous, boron, sulfur, or a halogen atom. Exemplary substituted hydrocarbyl moieties include, heterocyclo, alkoxyalkyl, alkenyloxyalkyl, alkynyloxyalkyl, aryloxyalkyl, hydroxyalkyl, protected hydroxyalkyl, keto, acyl, nitroalkyl, aminoalkyl, cyano, alkylthioalkyl, arylthioalkyl, ketals, acetals, amides, acids, esters, anhydrides, and the like. In a particular embodiment, R is a polyethylene oxide group.

Suitable monomers that can be used in the invention include but are not limited to one or more compounds with unsaturated double bonds such as methyl methacrylate, styrene, methyl methacrylate, methyl acrylate, ethylene diacrylate, ethylmethacrylate, acrylamide, diurethane dimethacrylate, poly-isoprene-graft-maleic acid monoethyl ester, glycidyl methacrylate, isocyanato-ethylmethacrylate, polyethylene glycol methacrylate, polyethylene glycol diacrylate, and/or polyethylene glycol dimethacrylate, preferably polyethylene glycol dimethacrylate. In particular embodiments, the monomers comprise one or more of isocyanato-ethylmethacrylate, glycidyl methacrylate, and polyethylene glycol diacrylate.

“Polymerizing agent” refers to a compound that is capable of initiating polymerization of monomers, preferably a radical polymerization initiator, to form a basecoat. Suitable polymerizing agents include but are not limited to azobis (cyanovaleric acid), azobiscyclohexanecarbonitrile, azobisisobutyronitrile (AIBN), benzoyl peroxide, iron (II) sulphate, and ammonium persulfate. The polymerizing agent may be designated A′, which in the context of a heterofunctional monomer of the Formula I, is capable of initiating a polymerization reaction with the A group of the Formula I.

“Portion” in reference to the coating of a polymeric surface, in particular a substrate, more particularly a medical device, means at least about 2%, 5%, 10%, 20%, 30%, 40%, 50%, 60%, 70%, 80%, 85%, 90%, 95%, or 100% of the polymeric surface is associated with a coating composition of the invention.

“Adhesive molecule” refers to a molecule that promotes cellular attachment or growth. Suitable adhesive molecules that may be used in the invention include fibronectin, laminin, vitronectin, thrombospondin, heparin-binding domains, and heparin sulfate binding domains, and synthetic polymers of amino acids containing adhesive sequences from one or more of the foregoing. Other suitable adhesive molecules include lectins that bind to heparin and carbohydrate moieties on the cell surface.

The term “about” includes plus or minus 0.1 to 50%, 5-50%, or 10-40%, preferably 10-20%, more preferably 10% or 15%, of the number to which reference is being made.

Coatings, Methods and Devices

The invention provides a coating composition for association with a polymeric surface comprising a basecoat displaying a plurality of active groups in association with serpins or serpin derivatives, wherein the serpins or serpin derivatives are not substantially cross-linked with other serpins or serpin derivatives. The association may involve non-covalent interactions such as electrostatic or hydrophobic interactions, van der Waal's forces, hydrogen bonding, or it may be a covalent interaction.

In a particular embodiment, the invention provides a coating composition for association with a polymeric surface comprising a basecoat comprising a polymer of heterofunctional monomers displaying a plurality of active groups in association with serpins or serpin derivatives, wherein the serpins or serpin derivatives are not substantially cross-linked with other serpins or serpin derivatives.

In an aspect, the invention provides a coating composition for a polymeric surface of a medical device which composition comprises a basecoat displaying a plurality of active groups in association with serpins or serpin derivatives, wherein the serpins or serpin derivatives are not substantially cross-linked with other serpins or serpin derivatives.

In embodiments, the serpin or serpin derivative is a complex or conjugate of heparin and antithrombin, in particular ATH.

A coating composition of the invention can alter the surface properties of a coated product, in particular, the serpin or serpin derivative can be an anticoagulant that provides anti-thrombogenic properties. In an embodiment, the coating provides a single layer of a serpin or serpin derivative on the surface of a medical device or product. In a further embodiment, the coating allows the production of a non-inflammatory material. In particular, the invention contemplates an anti-thrombogenic coating composition.

A coating composition of the invention may additionally comprise an adhesive molecule. In an embodiment, the serpin is a conjugate or complex comprising a serpin and heparin, and the adhesive molecule is bound to heparin. In a specific embodiment, the coating composition comprises a basecoat displaying a plurality of active groups in association with ATH molecules, wherein the heparin of the ATH molecules is associated with an adhesive molecule. Such coating composition is non-thrombogenic, and may in some applications promote cellular attachment and cell growth.

The association between the active groups and the serpin or serpin derivative (e.g. ATH) and optionally adhesive molecule in a coating composition of the invention may involve non-covalent interactions such as electrostatic or hydrophobic interactions, van der Waal's forces, hydrogen bonding, or it may be a covalent interaction.

A coating composition of the invention may include therein various conventional additives, including stabilizers, pH adjustment agents, and cosolvents. Additives are generally selected that are compatible with the intended use of the coating composition. Suitable additives to employ in coating compositions of the invention include benzalkonium, 4-dimethylaminopyridinium, tetrabutylammonium halides and the like.

The coating composition may be used to deliver other pharmaceutical and therapeutic agents including antibiotics and analgesics.

The invention also provides a method for coating a polymeric surface with a serpin or serpin derivative which comprises the following steps:
(i) introducing monomers with active groups on the polymeric surface; and
(ii) reacting with a preparation of the serpin or serpin derivative so that the serpin or serpin derivative associates with the active groups.

In an embodiment, monomers with active groups are introduced on the polymeric surface by applying a basecoat that displays a plurality of active groups on the polymeric surface. In an embodiment, the active groups are epoxy or epoxide groups.

In another embodiment, the monomers are covalently attached to the polymeric surface, and become part of the polymeric surface.

The invention also provides a method for coating a polymeric surface with a serpin or serpin derivative, which comprises the following steps:
(i) applying a basecoat to the polymeric surface where the basecoat displays a plurality of active groups; and
(ii) applying a preparation of a serpin or serpin derivative such that the serpin or serpin derivative associates with the active groups on the basecoat.

In an aspect the plurality of active groups are applied so that the active groups are not substantially cross-linked with other active groups in the basecoat.

In an embodiment, a method is provided for coating a polymeric surface with a serpin or serpin derivative, which comprises the following steps:
(a) applying monomers to the polymeric surface where the monomers comprise a plurality of active groups;
(b) allowing the monomers to cross-link to form a basecoat with active groups; and
(c) applying a preparation of the serpin or serpin derivative such that the serpin or serpin derivative associates with the active groups on the basecoat.

In accordance with an aspect of the invention, the method substantially provides a single layer of a serpin or serpin derivative on the basecoat where the serpin or serpin derivatives are not substantially cross-linked to other serpin or serpin derivatives. In particular, one embodiment of the present invention contemplates the attachment of one active group moiety to each single serpin molecule.

Thus, in an aspect, the invention provides a method for coating a polymeric surface with a serpin or serpin derivative, which comprises the following steps:
(i) applying monomers to the polymeric surface which monomers comprise a plurality of active groups;
(ii) allowing the monomers to cross-link to form a basecoat; and
(iii) applying a preparation of a serpin or serpin derivative such that the serpin or serpin derivative associates with an active group on the basecoat but does not substantially cross-link with another serpin or serpin derivative.

In another aspect, the invention provides a method for coating a substrate, in particular a medical device with a serpin or serpin derivative, which comprises the following steps:
(i) applying monomers to a portion of a polymeric surface of the substrate wherein the monomers comprise a plurality of active groups;
(ii) allowing the monomers to cross-link; and
(iii) applying a preparation of a serpin or serpin derivative such that the serpin or serpin derivative associates with an active group on the cross-linked basecoat but does not substantially cross-link with another serpin or serpin derivative.

A method of the invention for coating a polymeric surface in particular a polymeric surface of a substrate, more particularly a medical device may further comprise recovering any surplus serpin or serpin derivative that is not associated with the active groups on the cross-linked basecoat. Conventional techniques may be used to recover surplus serpin or serpin derivative.

In a further aspect the invention provides a method of applying a uniform coating to a medical device comprising providing a medical device comprising a polymeric surface, and applying a coating composition of the invention to a portion of the polymeric surface.

In aspects of the invention, the monomers are heterofunctional monomers of the Formula I:
Aₙ-(R)—Bₙ (Formula I)

wherein A is a group capable of polymerizing;

R is an optional linker;

B is an active group which, when the monomer has polymerized to form the cross linked basecoat, is capable of associating with the serpin or serpin derivative, and n is an integer, preferably 1-40, more preferably 1-20, still more preferably 1-10, most preferably 1-5, 1-3, and 1.

In a preferred embodiment, R is a C₁-C₅₀ divalent hydrocarbyl group, more preferably a polyethylene oxide group.

In an embodiment of a method of the invention, a basecoat is made by applying heterofunctional monomers of Formula I in combination with at least one polymerizing agent A′, wherein A′ is capable of initiating a polymerization reaction with the A-group of the heterofunctional monomer to form a cross-linked basecoat.

In a further embodiment, the B group is capable of forming a non-covalent association with a serpin or serpin derivative. In a particular embodiment, the association involves one or more of electrostatic or hydrophobic interactions, van der Waal's forces, and hydrogen bonding.

In a still further embodiment, the B group is capable of forming a covalent bond with a serpin or serpin derivative. In a particular embodiment, the covalent linkage involves a primary amino group on the serpin or serpin derivative.

In a particular embodiment of the invention, A and B are derived from one or more compounds, which are the same or different, including but not limited to acryloyl, methacryloyl, N-succinimidyl, sulfonylsuccinimidyl, glycidyl, 1,2-epoxy, chlorocarbonyl, and an anhydride functional group.

The monomers (basecoat) and preparation comprising a serpin or serpin derivative may be applied simultaneously, separately, sequentially in any order, and at different points in time, to a polymeric surface.

The methods of the invention are carried out under suitable conditions to provide the coating composition, or coated polymeric surface or medical device. It will be within the ordinary skill of a person skilled in the art to determine suitable reaction conditions including temperatures, amounts of monomers, serpin or serpin derivatives, reagents, and reaction times.

In aspects of the invention, the coating reaction can be performed at temperatures between about 0° to 80° C., in particular 20° to 60° C., and the reaction time can vary from about 5 minutes to 48 hours, in particular 20 min to 2 hours.

Monomers are preferably selected that provide a coating composition with a desirable graft density and/or durability.

In an embodiment, the monomers are glycidyl methacrylate monomers.

In another embodiment, the monomers are glycidyl methacrylate and polyethyleneglycol diacrylate, and preferably the volume of glycidyl methacrylate monomer to the volume of polyethyleneglycol methacrylate monomer in the basecoat is 3:1. A coating composition prepared using these monomers may be further characterized as providing a desirable graft density.

In a further embodiment, the monomers are polyethyleneglycol diacrylate, isocyanato-ethylmethacrylate monomer, and glycidyl methacrylate monomer, and preferably the volume of polyethyleneglycol diacrylate monomer to volume of isocyanato-ethylmethacrylate monomer to volume of glycidyl methacrylate monomer in the basecoat is 1 to 2 to 1.

The concentration of the monomers may be from 2% to 80% by volume, and preferably from about 10% to 50% by volume. The concentration of the serpin or serpin derivative may be from 0.01 mg/ml to 20 mg/ml by weight, and preferably from about 0.3 mg/ml to 8 mg/ml by weight.

In methods of the invention, the percent of total volume of all monomers in the total volume of monomers+solvent is 5-50%, 10-30%, or 20%.

In methods of the invention, the cross-linking of monomers can be achieved using a polymerizing agent. The concentration of polymerizing agent may be in the range of about 0.01% to about 5% by weight, and preferably in the range from about 0.05% to about 0.2% by weight. An annealing step under suitable conditions.(e.g. 50° C. for 30 minutes) may follow the cross-linling of the monomers.

In the methods of the invention, a serpin or serpin derivative can be applied in a solvent that is selected depending on the nature of the association between the active groups and serpin or serpin derivative. Suitable solvents are those that do not interfere with the activity of the serpin or serpin derivative. Examples of solvents include water (e.g. distilled, tap or the like), and organic solvents including but not limited to dichloromethane, chloroform, ethyl acetate, acetyl acetate, 1,4-dioxane, dimethylformamide, formamide, dimethylsulfoxide, tetrahydrofuran, acetone, methanol, ethanol, or a mixture of water and solvents including but not limited to dimethyl sulfoxide (DMSO), acetonitrile, alcohols such as methanol, ethanol, propanol, and ethylene glycol.

A method of the invention may also comprise attaching an adhesive molecule or pharmaceutic or therapeutic agent to a serpin or serpin derivative before or after applying the serpin or serpin derivative to the basecoat. In an embodiment, the serpin or serpin derivative is a conjugate or complex of heparin and antithrombin, in particular an ATH molecule, and the adhesive molecule is bound to heparin in the conjugate/complex or in the ATH molecule.

A method of the invention may further comprise analyzing the coating composition. Antithrombogenic properties may be determined by measuring the anti-factor Xa activity and anti-IIa activity. Coating uniformity may be analysed using conventional immunoassay procedures with antibodies specific for a serpin or serpin derivative. Coating stability and density may also be analyzed using standard methods such as those described in the Examples.

A method of the invention may also comprise the step of sterilizing a coated polymeric surface. Standard sterilization techniques can be employed in the invention (e.g. ethylene oxide).

The invention also contemplates a surface modification method based on single layer coating of a serpin or serpin derivative on a basecoat associated with a polymeric surface.

A coating composition of the invention can be applied to polymeric surfaces, in particular the blood-contacting, tissue-containing, or cell contacting surfaces of any of a wide variety of medical devices, to provide the medical devices with one or more non-thrombogenic surfaces. Coating compositions comprising adhesive molecules may also provide medical devices with one or more surfaces that promote cellular adhesion and attachment. A coating composition of the invention comprising an adhesive molecule can be used to attach cells to implantable medical devices such as prostheses, including vascular grafts, bone and cartilage implants, nerve guides and the like.

The invention also provides a polymeric surface that is coated with a cross-linked basecoat displaying a plurality of active groups associated with a serpin or serpin derivative, wherein the serpin or serpin derivative is associated with the plurality of active groups on the cross-linked basecoat. In a preferred embodiment, a polymeric surface is provided which is coated with a serpin or serpin derivative, wherein the serpin or serpin derivative is associated with a plurality of active groups on the cross-linked basecoat and serpin or serpin derivatives are not substantially cross-linked with other serpin or serpin derivatives.

The invention also contemplates a polymeric surface prepared by a method of the invention.

In an aspect, the invention provides a coated polymeric surface prepared by a method comprising:
(i) introducing monomers with active groups on a polymeric surface; and
(ii) reacting with a preparation of a serpin or serpin derivative so that the serpin or serpin derivative associates with the active groups.

A polymeric surface of the invention may additionally comprise an adhesive molecule associated with the serpin or serpin derivative.

The invention also contemplates a suitable substrate comprising a polymeric surface that includes on a portion thereof a coating comprising a cross-linked basecoat displaying a plurality of active groups capable of associating with a serpin or serpin derivative, wherein the serpin or serpin derivatives are not substantially cross-linked with other serpin or serpin derivatives.

In an aspect a medical device or product is provided comprising a polymeric surface that includes on a portion thereof a coating comprising a cross-linked basecoat displaying a plurality of active groups capable of associating with a serpin or serpin derivative, wherein the serpin or serpin derivatives are not substantially cross-linked with other serpin or serpin derivatives. In an embodiment, the medical device is designed to be at least partially inserted into a patient. A medical device of the invention may be sterilized using conventional methods known in the art (e.g. ethylene oxide).

In an aspect, the invention provides an antithrombotic medical material or device characterized in that it is a medical material or device having on a polymeric surface thereof, a coating composition of the invention. In an embodiment, the medical material or device additionally comprises an adhesive molecule, or a pharmaceutic or therapeutic agent. In a particular embodiment, a medical material or device is contemplated that is non-thrombogenic and promotes cellular adhesion.

The invention provides a medical device for the treatment of vascular disease comprising: a scaffold structure with a polymeric surface, and a coating composition associated with at least a portion of the polymeric surface.

In an embodiment, a catheter is provided comprising a substantially tubular body comprising a polymeric surface and a coating composition of the invention on a portion of the polymeric surface.

In a particular embodiment, the invention provides an intracorpeal medical device comprising a polymeric surface coated with a coating composition of the invention.

The invention also contemplates an implantable vascular device comprising a catheter or stent structure adapted for introduction into a vascular system of a patient the structure comprising a polymeric surface coated with a coating composition of the invention.

The invention also relates to a kit for preparing a coating composition or polymeric surface according to the invention. In an embodiment, the kit comprises:
(i) a preparation comprising a monomer as defined herein; and optionally
(ii) a preparation comprising a polymerizing agent as defined herein.

A kit of the invention may additionally comprise a preparation of the serpin or serpin derivative.

In an aspect of the invention, a method is provided for rendering a tissue- or blood-contacting surface of a medical device resistant to fibrin accumulation and clot formation which method comprises coating the surfaces with a non-thrombogenic coating composition of the invention.

In another aspect the invention contemplates a method of rendering a polymeric surface of a preformed medical material or device anti-thrombogenic comprising coating the polymeric surface with a coating composition of the invention.

A coating composition of the invention may be used to reduce clotting in a medical device used in a patient. In particular, the coating compositions can be used to reduce the thrombogenicity of internal and extracorporal devices that contact blood, and finds special use for coating thrombogenic medical devices including prosthetic surfaces.

The invention provides a method of treating a patient comprising introducing into the patient a medical device comprising a polymeric surface coated with a coating composition of the invention in an amount sufficient to prevent or inhibit thrombosis.

The present invention additionally provides methods of using a medical device coated with a coating composition of the invention. In an embodiment, the method comprises the steps of providing to a patient in need thereof a medical device comprising a body and at least a portion of the body coated with a coating composition comprising a cross-linked basecoat displaying a plurality of active groups capable of associating with a serpin or serpin derivative, wherein the serpin or serpin derivatives are not substantially cross-linked with other serpin or serpin derivatives.

The coating composition of the invention may have particular application in reducing or preventing vascular thrombosis associated with intravascular catheters. In an embodiment, a catheter coated with a coating composition of the invention comprising ATH is provided, wherein the patency of the catheter is at least 50, 75, or 100 days.

In an aspect the invention provides a method for, fractionating cells, in particular blood cells comprising applying the cells to a matrix coated with a coating composition of the invention.

The invention will be described in greater detail by way of specific examples. The following examples are offered for illustrative purposes, and are not intended to limit the invention in any manner. Those of skill in the art will readily recognize a variety of noncritical parameters that can be changed or modified to yield essentially the same results.

### EXAMPLE 1

Preparation of ATH

ATH was prepared using the method described in Chan et al, Journal of Biological Chemistry 272:22111-22117, 1997. In general, antithrombin and heparin in pH, 7.3 phosphate buffered saline (PBS) are mixed and incubated at 40° C. for 13 days. Sodium cyanoborohydride is added at the end of this incubation to ensure the covalent stability of any Shiff base that has not undergone an Amidori rearrangement. ATH and unreacted AT are then bound to a butyl hydrophobic interaction column to allow removal of unreacted heparin. After suitable high salt washes, the ATH and AT are released and bound to a DEAE anion exchange column. Unreacted AT is eluted with a low salt wash, while pure ATH is released with a high salt wash. The final product is dialyzed, concentrated, “sterilized” (when required), analysed for AT content, heparin content, ATH activity, formulated, and then aliquoted.

### EXAMPLE 2

ATH-Sparing Coating of Polyurethane Devices Using a Basecoat

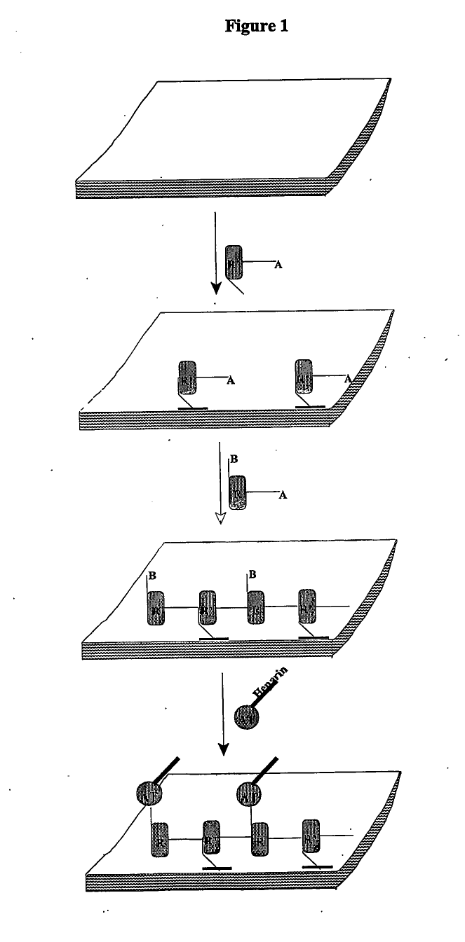

This example describes the procedure for coating ATH (antithrombin-heparin covalent complex) on polyurethane catheters via a basecoat that is attached to the polyurethane (FIG. 1). An improved chemistry is used that allows reuse of unbound coating ATH stock. The ATH and basecoat are linked to each other to form an ATH single-layer through a covalent linkage. This coating is considerably less expensive, inherently more uniform, more easily controlled, and the AT linker has greater exposure to blood.

Polyurethane catheters are dip-coated in isocyanato-ethylmethacrylate, reacted with the coating at 60° C. for 20 minutes, and then dip-coated in allyl glycidyl ether (epoxide) and the free radical initiator AIBN. Free radical polymerization to form the cross-linked basecoat occurs when the catheters are heated at 80° C. for 2 hours. This is followed by an annealing step carried out by lowering the temperature to 50° C. over a 30 minute period. The catheters are then immersed and incubated in a solution of ATH to generate the link between ATH and the basecoat. Unreacted ATH is recovered, the catheters are washed with 2% SDS in saline, and the catheters are sterilized with ethylene oxide. They are then spot-check analysed for AT content, anti-Xa activity, and for coating uniformity. The acrylate double bond is reactive in the presence of heat or light, thus all reactants and products must be protected from light. The epoxide is reactive to water, thus this group must be kept dry until it is exposed to ATH. The final graft density of ATH is dependant on the reaction concentration of ATH. The concentration of ATH that can be used is 1 mg/ml.

Detailed Procedures

1 ml isocyanato-ethylmethacrylate is added to 49 ml acetone and mixed. Uncoated catheters are immersed in the mixture, and, immediately after removal from the isocyanato-ethylmethacrylate acetone solution, incubated at 60° C. for 20 minutes. After incubation, any remaining solution (i.e. excess isocyanato-ethylmethacrylate) is discarded. Allyl glycidyl ether (20 ml) and 2,2′-azobis isobutyronitrile (AIBN) (0.025 gm) in acetone (30 ml) are added, mixed by inversion, and then incubated for 10 minutes at room temperature with inversion mixing. The liquid is discarded and the catheters are dried in a vacuum chamber for at least 4 hours at room temperature. The coating is polymerized at 80° C. for 40 minutes, and the temperature is reduced to 50° C. over a 20 minute period to anneal the coating.

The base coated catheters are immersed in ATH diluted with PBS to 50 ml of 1.0 mg (AT)/ml, and incubated at room temperature for at least 4 hours. The ATH solution is removed and is available to be used for coating other catheters. The catheters are washed with 0.125M saline+2% SDS at room temperature for 30 minutes. The solution is discarded and the wash step is repeated. The catheters are washed with PBS and Milli-Q water, and dried with a clean nitrogen gas stream.

### EXAMPLE 3

ATH Sparing Non-Covalent Coating of Polyurethane Devices

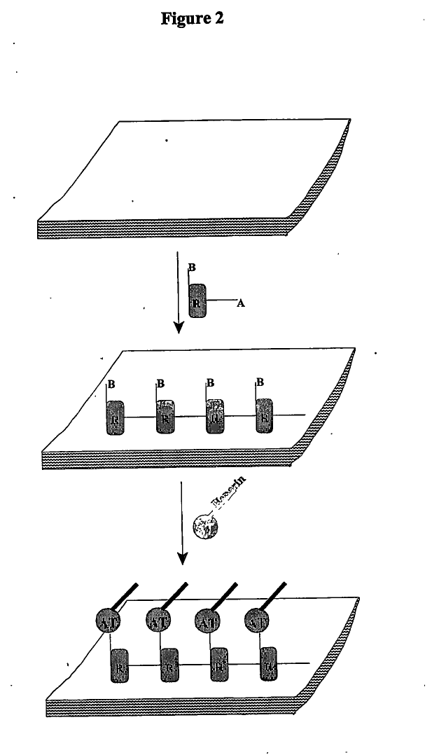

ATH (antithrombin-heparin covalent complex) may be coated on polyurethane catheters via a non-covalently attached basecoat sheath using a chemistry that allows reuse of unbound coating ATH stock (FIG. 2). The ATH and sheath are covalently linked to each other as ATH (single-layer) on a basecoat (cross-linked complex). This coating is considerably less expensive, inherently more uniform, more easily controlled, and the AT linker has greater exposure to blood.

Polyurethane catheters are dip-coated in a mixture of glycidyl methacrylate, polyethylene glycol diacrylate, and the free radical initiator AIBN in acetone. The coating is dried in place and polymerized into a cross-linked basecoat by heating at 80° C. for 40 minutes, followed by cooling to 50° C. over 20 minutes. The catheters are then immersed and incubated in a solution of ATH at room temperature for 20 hours to generate the covalent (epoxide-primary amine) link Unreacted ATH is recovered, the catheters are thoroughly sequentially washed with several solutions, and the catheters are sterilized with ethylene oxide. The catheters can be analyzed for AT content, anti-Xa activity, and coating uniformity.

Detailed Procedure

A basecoat solution is prepared by mixing glycidyl methacrylate (10.5 ml), polyethylene glycol diacrylate (3.5 ml), 2,2″-azobisisobutyronitrile (AIBN) (0.035 gm), and acetone (56 ml). Uncoated catheters are totally immersed in the basecoat solution, incubated at room temperature for 20 minutes, and the liquid is discarded. The catheters are dried in a vacuum chamber for 2 hours at room temperature, and the coating is polymerized by heating at 80° C. for 40 minutes. The temperature is reduced to 50° C. over a 20 minute period to anneal the coating.

Base-coated catheters are immersed in ATH diluted with Milli-Q water to 70 ml of 1.0 mg (AT)/ml, and incubated at room temperature with stirring (overnight or for at least 4 hours). The ATH solution is removed for use in coating other catheters. The catheters are flushed with 0.15 M phosphate buffer, buffer+2M NaCl, buffer+0.1% SDS, PBS, and Milli-Q water. The catheters are dried with clean nitrogen gas stream, and sterilized (e.g. by ethylene oxide).

### EXAMPLE 4

ATH-Coating of Polyurethane Catheters

This example describes the procedure for coating ATH (antithrombin-heparin covalent complex) on polyurethane catheters via a basecoat that is covalently attached to the polyurethane. An improved chemistry is used that allows reuse of unbound coating ATH stock. The ATH and basecoat are covalently linked to each other to form an ATH single-layer. This coating is considerably less expensive, inherently more uniform, more easily controlled, and the AT linker has greater exposure to blood.

Polyurethane catheters are immersed for 60 minutes at 40° C. in isocyanato-ethylmethacrylate (dissolved in acetone) to form a covalent bond between the isocyanato group and nitrogen atoms in polyurethane. The catheters are then immersed for 10 minutes at room temperature in allyl glycidyl ether (epoxide, dissolved in acetone) with the free radical initiator AIBN. Free radical polymerisation to form progressive cross-links between vinyl groups occurs when the catheters are heated to 80° C. for 2 hours, followed by an annealing step at 50° C. for 30 minutes. The catheters are then immersed and incubated in a solution of ATH to generate the covalent link between ATH primary amines and basecoat epoxide groups. Unreacted ATH is recovered. The catheters are washed with several solutions and sterilized with ethylene oxide. Catheters are then spot-check analysed for AT content, anti-Xa activity, and for coating uniformity. The acrylate double bond is reactive in the presence of heat or light, thus all reactants and products are protected from light. The epoxide is reactive to water, thus this group is kept dry until it is exposed to ATH. The final graft density of ATH correlates with the reaction concentration of ATH (in particular, 1 mg/ml is used).

### EXAMPLE 5

Catheter occlusion and vascular thrombosis are common problems associated with use of intravascular catheters. The types of proteins adsorbed onto biomaterials affects thrombus formation at the blood-material interface. Since the outer surface of an implanted catheter is exposed to flowing blood and the inner surface of the catheter is exposed to different fluids (including, saline, drugs being infused as well as blood), protein adsorption on the outer and inner surface of a catheter is potentially different, with associated different effects on thrombogenicity. Therefore, it is important to establish whether protein adsorption is indeed different on the inside and outside of a catheter in vivo.

In the present study protein adsorption patterns were investigated on the inside and outside surfaces of polyurethane catheters coated with a novel covalent antithrombin-heparin complex (ATH) (Chan A K C, et al, J Biol Chem, 272, 22111, 1997), and used in a rabbit model for 106 days. ATH coated surfaces have previously been shown to be resistant to thrombus formation (Klement P, et al, Biomaterials, 23, 527, 2000), but in this study the ATH coated catheters were tested for a long period of time and the resulting effects on outer and inner catheter surfaces were compared.

Materials and Methods:

ATH, prepared according to protocols published previously (Chan A K C, et al, J Biol Chem, 272, 22111, 1997), was purified by hydrophobic chromatography on butyl Sepharose followed by anion exchange chromatography on DEAE Sepharose. ATH was concentrated at 4° C. by pressure dialysis under nitrogen.

Polyurethane catheters were coated on both the inner and outer surfaces by polymerization of an activated monomer. ATH was then covalently linked to the surface by incubation with the polymerized, activated monomer on the catheters. The coated catheters were rinsed sequentially with buffer (pH 8.0), followed by 2M NaCl in buffer, then SDS in buffer and finally PBS. Catheters were allowed to drain, placed in semi permeable bags, sterilized with ethylene oxide, and stored dry at room temperature prior to implantation into the animal. The graft density of the ATH on the catheters was 5-10 pmol/cm².

New Zealand white male rabbits were anaesthetized, and the coated catheters inserted into the right jugular vein and advanced to the edge of the right atrium. The rabbits were allowed to recover. Catheter patency was examined by withdrawing 0.5 mL blood samples through the catheter twice daily, followed by a 2 mL saline flush of the catheter. At the end of the experiment (determined by catheter occlusion or predetermined time), the coated catheter was removed from the animal and rinsed with saline. Adsorbed proteins were eluted from the inner and outer surfaces of the catheters with 2% SDS. Initially only the inner surfaces were exposed to the SDS solution. The catheter was then completely drained of SDS, cut into 0.5 cm lengths, and the proteins eluted from the outer surface by exposure to 2% SDS. Reduced SDS-PAGE gels (12%) and immunoblots were then run on the eluted protein samples according to protocols published previously (Cornelius R M, et al, J Biomed Mater Res, 60, 622, 2002). Antibodies used in the immunoblotting procedures were directed against the following proteins: prekallikrein, fibrinogen, antithrombin (AT), plasminogen, α-2-macroglobulin, thrombin, heparin cofactor II and vitronectin.

Results and Discussion:

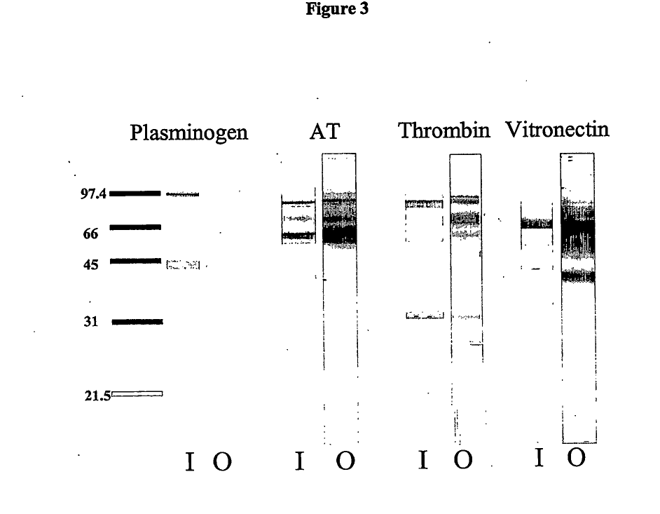

The ATH coated catheters in this study remained patent at 106 days (n=2). In contrast, the uncoated PU catheters occluded within 12 days as previously reported (n=8). Immunoblots of proteins eluted from the inner (I) and outer (O) surfaces of the PU-ATH catheters are shown in FIG. 3. All proteins probed for were detected, although the intensities of the bands for prekallikrein (not shown) and plasminogen were weak. A strong response was obtained for AT as expected, with strong bands at ˜59 kDa (AT) and weaker bands at ˜95 kDa (thrombin-AT complex). Unmodified PU control surfaces, tested previously in the rabbit model, did not bind AT (Du Y J, et al, Trans Soc Biomater, 25, 404, 2002). The increased absorption of AT on ATH coated catheters compared to controls demonstrated the presence of heparin on ATH coated catheters because of the high affinity of AT to the heparin portion of ATH. A positive response was also obtained for thrombin with bands at 35 kDa (thrombin) and ˜95 kDa (thrombin-AT complex). Strong responses were also obtained for albumin (not shown) and vitronectin. Although intensities differed, it is interesting to note the similarity in banding patterns obtained on the inner and outer surfaces of the catheters for a given protein. Stronger immunoblotting responses were obtained for AT, thrombin, and vitronectin on the outer surfaces of the catheters, while weaker responses were obtained for plasminogen and heparin cofactor II on the outer surfaces.

These data suggest that greater amounts of some proteins (i.e. AT, thrombin, vitronectin) may be present on the outer surfaces as compared to the inner surfaces of the catheters. The experimental design was such that the inner surfaces of the catheters were fully exposed to blood twice daily. The outer surface was likely in direct contact with blood for the first few days of the experiment, but contact with the blood may have altered over time depending on the interaction between the catheter and blood vessel.

It is concluded that both the inner and outer surfaces of ATH coated catheters bound AT in large quantities. Prekallikrein, plasminogen, α-2-macroglobulin, heparin cofactor II and vitronectin, were also detected in varying amounts on the catheters. However the quantities of these proteins were different on the inner and outer surfaces.

### EXAMPLE 6

Effect of Monomer Composition in Basecoat on ATH Coating of Polymeric Surfaces

Experiments were devised to determine the effect of varying ratios of poly(ethyleneglycol)diacrylate (monomer with A group only) to isocyanato-ethylmethacrylate (monomer for covalent linkage to polymeric surface) to glycidyl methacrylate (monomer with both A and B groups) and varying % total monomer concentrations (100×[(sum of the volume of all monomers added)/(volume of monomers+volume of solvent)]) on final graft density of ATH coated on polyurethane catheters. Poly(ethyleneglycol)diacrylate, isocyanato-ethylmethacrylate and glycidyl methacrylate were mixed with 2,2′-azobis isobutyronitrile (AIBN) polymerizing agent in acetone solvent and incubated with polyurethane catheter segments for 20 minutes at room temperature. To form the basecoat, the segments were then gravity drained of monomer solution, vacuum dried and incubated at 80° C. for 40 minutes, followed by cooling to 50° C. over 20 minutes. The catheters are then immersed in a solution of ATH (containing ATH labelled with ¹²⁵I) and incubated at room temperature for 20 hours to generate the covalent link between ether groups on the basecoat and amino groups on ATH. The volume ratio of the 3 monomers was varied and the total concentration of the monomers in acetone was varied to determine the effect of monomer composition in the basecoat on density of ATH grafted onto the polymeric surface (polyurethane catheter segment). Detection of ATH present on the surface was by gamma counting of the catheter segments to measure remaining surface-bound ¹²⁵I-ATH. Stability of ATH coating was assessed by multiple washes and protease treatment.

Detailed Procedure

Various volume ratios and total concentrations of monomers were tested to determine the effect of basecoat composition on ATH attachment on polyurethane catheter surfaces. An example procedure is given below for reaction of a 1:1:2 volume ratio of poly(ethyleneglycol)diacrylate to isocyanato-ethylmethacrylate to glycidyl methacrylate at a total volume of 20 ml of monomers per 100 ml of monomers+acetone solvent (20% monomers by volume). A basecoat solution was prepared by mixing poly(ethyleneglycol)diacrylate (0.2 ml), isocyanato-ethylmethacrylate (0.2 ml), glycidyl methacrylate (0.4 ml), 0.002 g 2,2″-azobisisobutyronitrile (AIBN), and acetone (3.2 ml). Uncoated polyurethane catheter segments (7 French, 1 cm² surface area, approximately 1 cm in length) were totally immersed in the basecoat, and incubated at room temperature for 20 minutes. The liquid was discarded. The catheters were dried in a vacuum chamber for 2 hours at room temperature, and the coating polymerized by heating at 80° C. for 40 minutes. The temperature was reduced to 50° C. over a 20 minute period to anneal the coating.

Base coated catheter segments were immersed in ATH solution and incubated for 20 hours at room temperature. The ATH solution contained ATH that had been labelled using Na¹²⁵I (New England Nuclear) and iodobeads (Pierce Chemical Company) according to the method by the manufacturer of the iodobeads. The ¹²⁵I-ATH incubation solution was 1.0 mg (AT)/ml of PBS and had a gamma radioactivity of 26300 counts per minute per ml. The ATH solution was removed for use in coating of other catheters. The catheter segments were washed by agitation. In some cases, the catheters were each washed with 5 ml of 0.8 g NaCl/100 ml H₂O for 24 hours. After 24 hours of NaCl wash, the wash solution was replaced every 24 hours with another 5 ml 0.8 g NaCl/100 ml H₂O and washing continued. After every change of wash solution, the catheter segment was gamma counted for remaining bound ¹²⁵I-ATH. In other cases, the catheters were given 3 short washes with 5 mL of 0.8 g NaCl/100 ml H₂O, followed by a wash with ml of 2% SDS in H₂O for 24 hours. After 24 hours of SDS wash, the wash solution was replaced every 24 hours with another 1 ml of 2% SDS, along with a gamma count of remaining bound ¹²⁵I-ATH. Stability of coating to protease treatment of surfaces washed with 0.8 g NaCl/100 ml was evaluated. Catheter segments washed with NaCl solution were agitated with 1 ml solution of a general protease (P-5147 from Sigma) at 0.1 mg protease/ml of H₂O for 24 hours at room temperature. Every 24 hours, the protease solution was replaced with a fresh 1 ml of 0.1 mg protease/ml of H₂O and the catheter segment gamma counted to determine remaining bound ¹²⁵I-ATH. Given the gamma radioactivity per mg ATH in the original incubation mixtures and the surface area of the catheter segments, the graft density of ATH on each catheter in pmoles/cm² was calculated.

Results

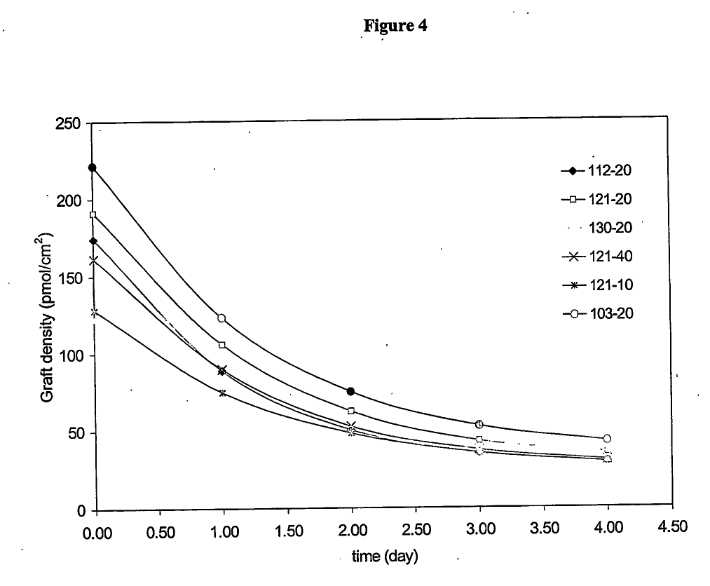

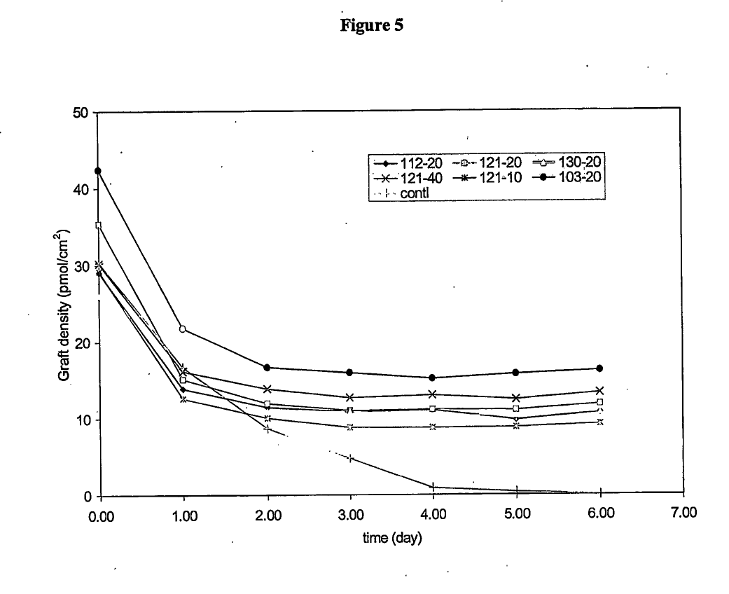

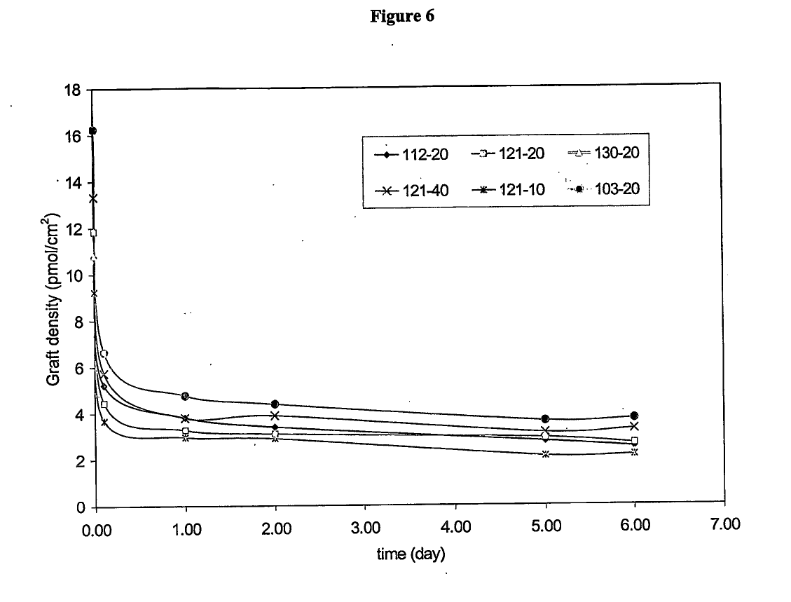

The effect of varying monomer composition and total monomer concentration on graft density of ATH coated onto catheters during washing with 0.8 g NaCl/100 ml H₂O is shown in FIG. 4. Graft density plateaued after 3 changes of wash solution. Out of all the combinations of monomer compositions and total concentrations tested, the highest graft density was observed with a volume ratio of poly(ethyleneglycol)diacrylate to isocyanato-ethylmethacrylate to glycidyl methacrylate=1:0:3 and a % total volume of monomers/total volume of monomers+solvent=20%. The effect of varying monomer composition and total monomer concentration on graft density of ATH coated onto catheters during washing with 2% SDS is shown in FIG. 5. Again, graft density plateaued after 3 changes of wash solution. Also, out of all the combinations of monomer compositions and total concentrations tested, the highest graft density was observed with a volume ratio of poly(ethyleneglycol)diacrylate to isocyanato-ethylmethacrylate to glycidyl methacrylate=1:0:3 and a % total volume of monomers/total volume of monomers+solvent=20%. Data for the effect of protease treatment on ATH graft density is shown in FIG. 6. ATH was rapidly lost from the catheters during the first 24 hour incubation with protease, after which the amount of ATH bound to the catheters remained fairly constant with continuing protease incubations, regardless of the monomer composition in the basecoat.

The present invention is not to be limited in scope by the specific embodiments described herein, since such embodiments are intended as but single illustrations of one aspect of the invention and any functionally equivalent embodiments are within the scope of this invention. Indeed, various modifications of the invention in addition to those shown and described herein will become apparent to those skilled in the art from the foregoing description and accompanying drawings. Such modifications are intended to fall within the scope of the appended claims.

All publications, patents and patent applications referred to herein are incorporated by reference in their entirety to the same extent as if each individual publication, patent or patent application was specifically and individually indicated to be incorporated by reference in its entirety. All publications, patents and patent applications mentioned herein are incorporated herein by reference for the purpose of describing and disclosing the domains, cell lines, vectors, methodologies etc. which are reported therein which might be used in connection with the invention. Nothing herein is to be construed as an admission that the invention is not entitled to antedate such disclosure by virtue of prior invention.

It must be noted that as used herein and in the appended claims, the singular forms “a”, “an”, and “the” include plural reference unless the context clearly dictates otherwise. Thus, for example, reference to “a host cell” includes a plurality of such host cells, reference to the “antibody” is a reference to one or more antibodies and equivalents thereof known to those skilled in the art, and so forth.

Below full citations are set out for the references referred to in the specification.

1. A coating composition for association with a polymeric surface comprising a cross-linked basecoat displaying a plurality of active groups in association with serpin or serpin derivatives, wherein the serpin or serpin derivatives are not substantially cross-linked with other serpin or serpin derivatives.

2-39. (canceled)
